(19) United States
(12) Patent Application Publication
(10) Pub. No.: US 2003/0035551 A1
(43) Pub. Date: Feb. 20, 2003

(54) Ambient-aware headset

(76) Inventors: John J. Light, Beaverton, OR (US); Michael D. Smith, Hillsboro, OR (US); Dhananjay V. Keskar, Beaverton, OR (US)

Correspondence Address: James H. Salter, BLAKELY, SOKOLOFF, TAYLOR & ZAFMAN LLP, Seventh Floor, 12400 Wilshire Boulevard, Los Angeles, CA, 90025-1026, US

(21) Appl. No.: 09/933,626

(22) Filed: Aug. 20, 2001

(51) Int. Cl. A61F 11/06; G10K 11/16; H03B 29/00

(52) U.S. Cl. 381/71.6; 381/74; 381/94.8

(57) ABSTRACT

An ambient-aware headset is described. A mixer coupled to an external audio source receives an external audio signal. The mixer is also coupled to an ambient audio source to receive an ambient audio signal. The mixer mixes the external audio signal and the ambient audio signal according to a specified relationship. A speaker coupled to the mixer emits the external audio signal and the ambient audio signal into an ear canal of a user after the external audio signal and the ambient audio signal have been mixed by the mixer.

### FIELD OF THE INVENTION

The field of the invention relates generally to a headset and specifically to a headset having a mixer to process ambient sound.

### BACKGROUND

Headsets and earphones are becoming increasingly popular with the mushrooming in the number and type of portable electronic devices. A headset eliminates the need of the headset wearer to hold a speaker, such as a telephone “receiver” to his ear in order to hear the audio sound or voice being emitted by the speaker. The wearer can thus listen to audio emitted from an electronic device while keeping one or both hands free to do other things.

One problem, however, with conventional headsets is that they do not restrict outside or “ambient” noise from being heard by the headset wearer. In an attempt to solve this problem, various types of “noise cancellation” headphones have been produced. Noise cancellation headphones generate “anti-noise” waves to “cancel” ambient noise, such as airplane cabin noise. Such headphones can allow the wearer to simultaneously listen to another audio source, such as a personal CD player, through the headphones and also to adjust ambient noise reduction. However, noise cancellation headphones should not be used in certain situations because of the potential dangers of the headset wearer not hearing ambient noise adequately, such as in the case of driving an automobile. If, on the other hand, the headset wearer opts to reduce the level of ambient noise reduction so that he may better hear ambient noise, then, the headset may not filter ambient sounds sufficiently for such purposes as carrying on a wireless telephone conversation. Thus, using a state of the art headset, the headset wearer is limited in how and where he can use his headset.

### BRIEF DESCRIPTION OF THE DRAWINGS

FIG. 1 is a simplified block diagram of one embodiment of an ambient-aware headset according to the present invention.

FIG. 2 is a block diagram of another embodiment of an ambient-aware headset.

FIG. 3 is a flow diagram of one embodiment of a method for mixing an external audio signal and an ambient audio signal according to a specified relationship.

FIG. 4 is a flow diagram of another embodiment of a method for mixing an external audio signal and an ambient audio signal according to a specified relationship.

FIG. 5 is a diagram of a system-level overview of an embodiment of the invention.

FIG. 6 is a diagram of an operating environment suitable for practicing the invention.

FIG. 7 is a diagram of a computer-readable media, which may be used within an operating environment, such as the environment of FIG. 6, according to one embodiment of the present invention.

### DETAILED DESCRIPTION

An ambient-aware headset is described. In the following description, for purposes of explanation, numerous specific details are set forth in order to provide a thorough understanding of the invention. However, it will be apparent to one with ordinary skill in the art that these specific details need not be used to practice the present invention. In other instances, well-known structures and devices are shown in block diagram form in order to avoid unnecessarily obscuring the present invention.

A mixer coupled to an external audio source receives an external audio signal. The mixer is also coupled to an ambient audio source to receive an ambient audio signal. The mixer mixes the external audio signal and the ambient audio signal according to a specified relationship. A speaker coupled to the mixer emits the external audio signal and the ambient audio signal into an ear canal of a user after the external audio signal and the ambient audio signal have been mixed by the mixer. An ear canal is a narrow tube like passage through which sound enters the ear. Use of an ambient aware headset may enable a headset wearer to interact more seamlessly between physical space and cyberspace. The headset will allow the headset wearer to select the degree to which he will participate in an activity in physical space as opposed to an activity in cyberspace.

The term “headset” means a device consisting of one or more earphones with a headband for holding them over the ears and sometimes with a mouthpiece attached. It will be appreciated by one skilled in the art that there is no particular physical configuration for the headset described herein. Rather, any configuration which enables the headset wearer to hear an incoming audio signal is contemplated. Such configurations are not limited to those having a headband; a single earpiece inserted directly into the ear of the wearer is also contemplated. The term “user” is used interchangeably with the terms “wearer” and “headset wearer.”

The term “ambient” means of the surrounding area or environment. Thus, “ambient sound” refers to sounds or noises audible within the area surrounding the headset wearer. Ambient sound includes voices and background noise, but the term is not so limited.

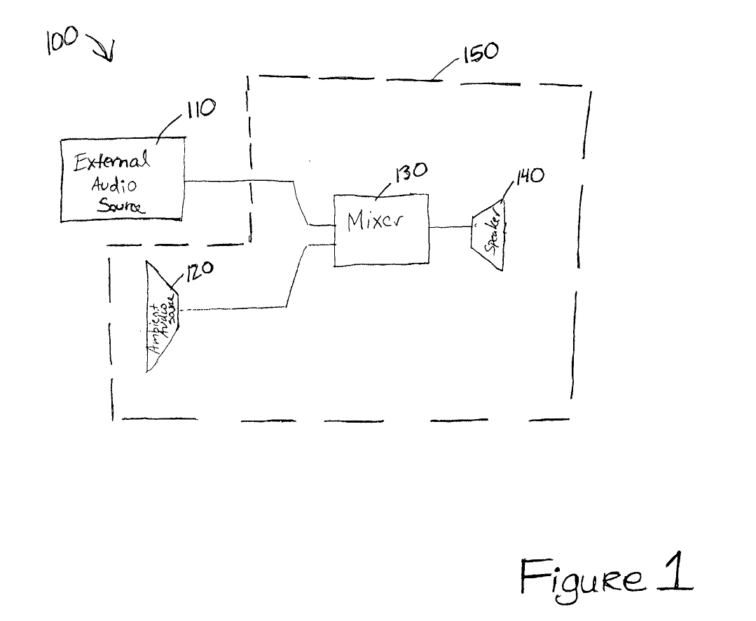

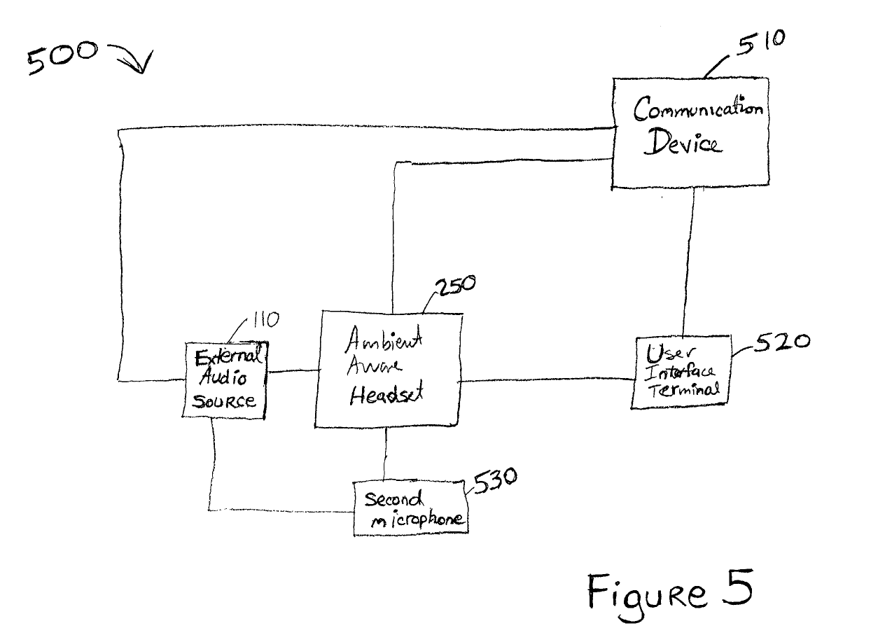

FIG. 1 is a simplified block diagram of one embodiment of an ambient-aware headset according to the present invention. It will be appreciated that the headset may operate in the context of network environment, such as the environment of FIG. 5. In one embodiment, headset 150 is comprised of mixer 130, ambient audio source 120 and speaker 140. External audio source 110 is coupled to mixer 130. External audio source may be a telephone, a audio playing device or a personal electronic device, but the invention is not so limited. Ambient audio source 120 is also coupled to mixer 130. Ambient audio source 120 may be a microphone to capture ambient sound, but the invention is not so limited. Mixer 130 mixes an external audio signal generated by external audio source 110 and an ambient audio signal communicated ambient audio source 120 to mixer 130 according to a specified relationship. In one embodiment, the specified relationship is determined by a preference input by the user. In another embodiment, mixer 130 further comprises a digital signal processor (DSP) to process the ambient audio signal and external audio signal digitally. Mixer 130 is further coupled to speaker 140. Speaker 140 emits the external audio signal and the ambient audio signal into an ear canal of a user after the external audio signal and the ambient audio signal have been mixed by the mixer 130. In one embodiment, ambient audio source 120, mixer 130, and speaker 140 are an integrated assembly.

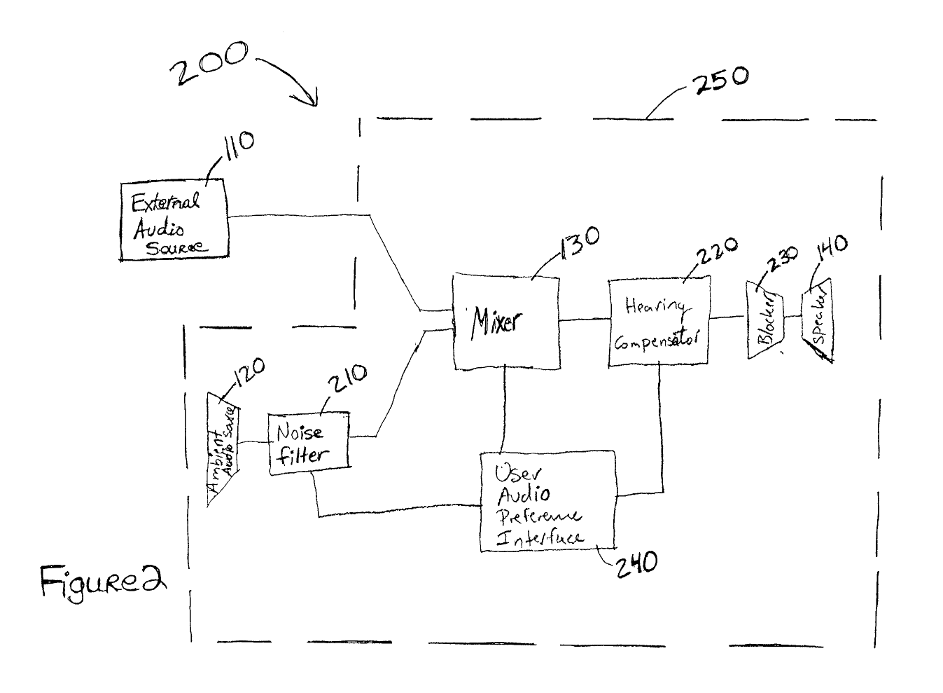

FIG. 2 is a block diagram of another embodiment of an ambient-aware headset. In one embodiment, headset 250 is composed of mixer 130, ambient audio source 120, noise filter 210, hearing compensator 220, blocker 230, speaker 140 and user audio preference interface (UAPI) 240. Noise filter 210 is coupled to ambient audio source 120 to filter noise from the ambient audio signal. In one embodiment, the noise is filtered according to an audio preference asserted by the user. The audio preference may be asserted by the user at a user audio preference interface (UAPI) 240. In another embodiment, the UAPI 240 is coupled to the mixer 130 to receive an audio preference from the user, the audio preference being used to determine the specified relationship according to which the external audio signal and the ambient audio signal are mixed by mixer 130. Hearing compensator 220 is coupled to mixer 130. Hearing compensator 220 compensates for a hearing defect of the user. In one embodiment, the hearing defect is compensated for according to an audio preference asserted by the user. The audio preference may be asserted by the user at the UAPI 240. Blocker 230 is coupled to speaker 140. Blocker 230 prevents entrance of the ambient audio signal that was not mixed by the mixer from entering the ear canal of the user. In one embodiment, blocker 230 is a component of speaker 140.

In one embodiment where the mixer 130 further comprises a digital signal processor to process the ambient audio signal and the external audio signal digitally, the DSP may have a noise filter to filter noise coupled to the ambient audio source 120 to filter noise from the ambient audio signal and/or a hearing compensator coupled to the mixer 130 to compensate for a hearing defect of the user.

In one embodiment, the headset further comprises a second microphone coupled to the external audio source for transmitting an outgoing audio signal from the user to the external audio source. For example, the headset may have a microphone for capturing the speech of the headset wearer in a wireless telephone conversation.

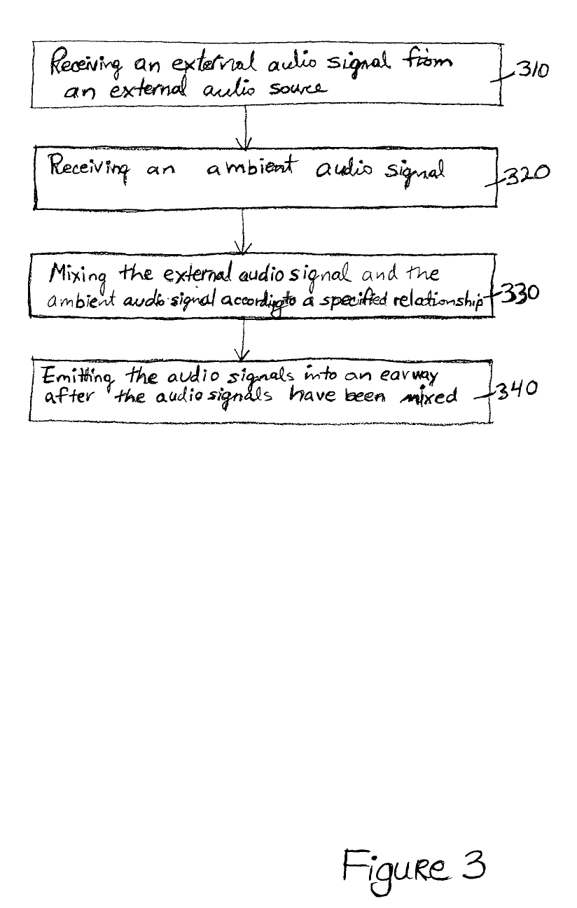

FIG. 3 is a flow diagram of one embodiment of a method for mixing an external audio signal and an ambient audio signal according to a specified relationship. At block 310, an external audio signal is received from an external audio source. At block 320, an ambient audio signal is received. At block 330, the external audio signal and ambient audio signal are mixed according to a specified relationship. In one embodiment, the specified relationship is pre-determined by a preference input by a user. At block 340, the external audio signal and the ambient audio signal are emitted into the ear canal of a user after the external audio signal and the ambient audio signal have been mixed according to the specified relationship.

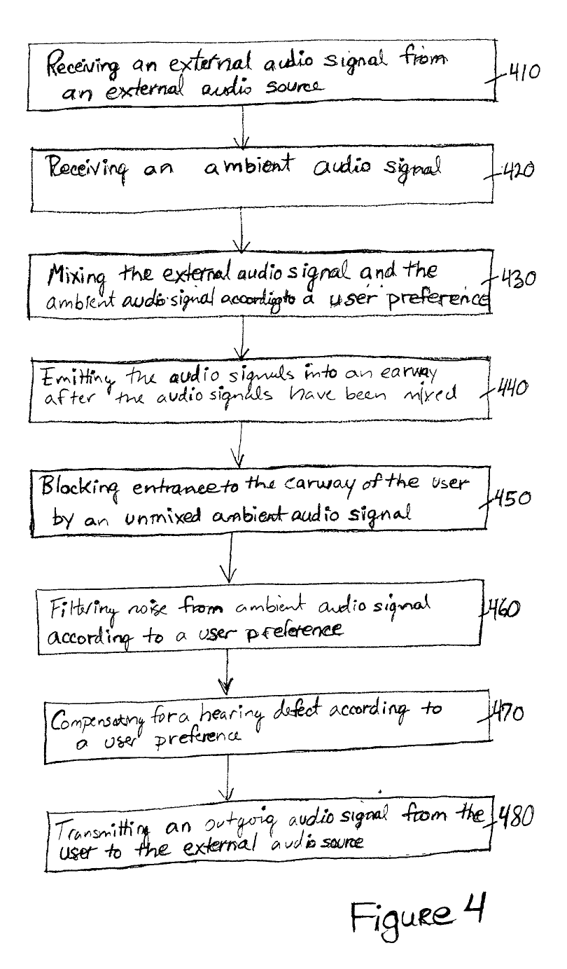

FIG. 4 is a flow diagram of another embodiment of a method for mixing an external audio signal and an ambient audio signal according to a specified relationship. At block 410, an external audio signal is received from an external audio source. At block 420, an ambient audio signal is received. At block 430, the external audio signal and ambient audio signal are mixed according to a specified relationship. In one embodiment, the specified relationship is pre-determined by a preference input by a user. At block 440, the external audio signal and the ambient audio signal are emitted into the ear canal of the user after the external audio signal and the ambient audio signal have been mixed according to the specified relationship. At block 450, entrance to the ear canal of the user by the ambient audio signal that has not been mixed according to the specified relationship is blocked. At block 460, noise is filtered from the ambient audio signal. In one embodiment, the filtering is performed according to a preference input by the user. This preference may be pre-defined by the user. At block 470, compensation is made for a hearing defect of the user. In one embodiment, the compensation is performed according to a preference input by the user. In another embodiment, the compensation is performed according to a preference input by a medical professional. At block 480, an outgoing audio signal is transmitted from the user to the external audio source.

In one embodiment, the mixing further comprises mixing in digital form using a digital signal processor (DSP). In another embodiment, the noise filtering is performed by a DSP. In yet another embodiment, the hearing compensation is performed by the DSP.

It will be appreciated that the preferences used to modify the audio system before it enters the ear canal may be pre-defined by the headset wearer or any other person.

FIG. 5 is a diagram of a system-level overview of an embodiment of the invention. Ambient-aware headset 250 is coupled to external audio source 110 to receive an external audio signal. Ambient-aware headset 250 also receives an ambient audio signal. Ambient-aware headset 250 is also coupled to second microphone 530 to transmit an outgoing audio signal from the headset wearer to external audio source 110. Communication device 510 is communicatively coupled to external audio source 110 and ambient-aware headset 250 to communicate audio data. Communication device 510 may be a wireless telephone transmission tower or a wireless audio data transmission tower, but the invention is not so limited. Communication device 510 and ambient-aware headset 250 may also be communicatively coupled to user interface terminal 520. User interface terminal 520 may store or relay audio data between communication device 510 and ambient-aware headset 250. User interface terminal 520 may be a personal computer, but the invention is not so limited.

In one embodiment, a user wearing headset 250 carries on a wireless telephone conversation in which the incoming voice is received at headset 250 via external audio source 110 where external audio source 110 is a wireless phone. The user's voice is captured by second microphone 530 and communicated to external audio source 110. External audio source 110 communicates incoming and outgoing voice streams with communication device 510.

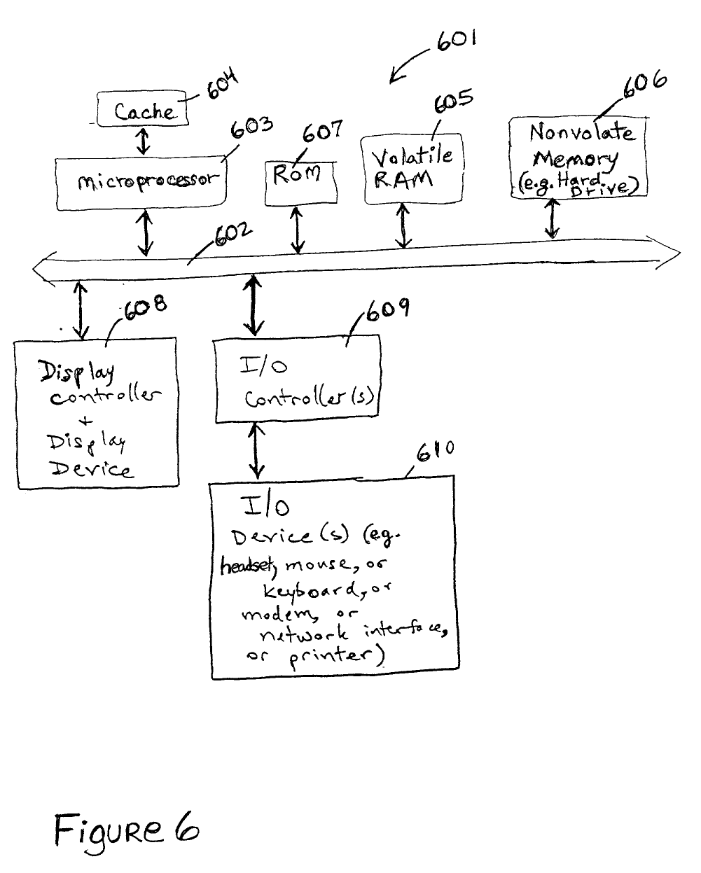

FIG. 6 shows one example of a typical computer system which may be used with the present invention. Note that while FIG. 6 illustrates various components of a computer system, it is not intended to represent any particular architecture or manner of interconnecting the components as such details are not germane to the present invention. It will also be appreciated that network computers and other data processing systems which have fewer components or perhaps more components may also be used with the present invention.

As shown in FIG. 6, the computer system 601, which is a form of a data processing system, includes a bus 602 which is coupled to a microprocessor 603 and a ROM 607 and volatile RAM 605 and a non-volatile memory 606. The microprocessor 603 is coupled to cache memory 604 as shown in the example of FIG. 6. The bus 602 interconnects these various components together and also interconnects these components 603, 604, 605, and 606 to a display controller and display device 608 and to peripheral devices such as input/output (I/O) devices which may be ambient-aware headsets, mice, keyboards, modems, network interfaces, printers and other devices which are well known in the art. Typically, the input/output devices 610 are coupled to the system through input/output controllers 609. The volatile RAM 605 is typically implemented as dynamic RAM (DRAM) which requires power continually in order to refresh or maintain the data in the memory. The non-volatile memory 606 is typically a magnetic hard drive or a magnetic optical drive or an optical drive or a DVD RAM or other types of memory systems which maintain data even after power is removed from the system. Typically, the non-volatile memory will also be a random access memory although this is not required. While FIG. 6 shows that the non-volatile memory 606 is a local device coupled directly to the rest of the components in the data processing system, it will be appreciated that the present invention may utilize a non-volatile memory which is remote from the system, such as a network storage device which is coupled to the data processing system through a network interface such as a modem or Ethernet interface. The bus 602 may include one or more buses connected to each other through various bridges, controllers and/or adapters as is well known in the art. In one embodiment the I/O controller 609 includes a USB (Universal Serial Bus) adapter for controlling USB peripherals.

It will be apparent from this description that aspects of the present invention may be embodied, at least in part, in machine-executable instructions, e.g. software. That is, the techniques may be carried out in a computer system or other data processing system in response to its processor, such as a microprocessor, executing sequences of instructions contained in a memory, such as ROM 607, volatile RAM 605, non-volatile memory 606, cache 604 or a remote storage device. In various embodiments, hardwired circuitry may be used in combination with software instructions to implement the present invention. Thus, the techniques are not limited to any specific combination of hardware circuitry and software nor to any particular source for the instructions executed by the data processing system. In addition, throughout this description, various functions and operations are described as being performed by or caused by software code to simplify description. However, those skilled in the art will recognize what is meant by such expressions is that the functions result from execution of the code by a processor, such as the microprocessor 603.

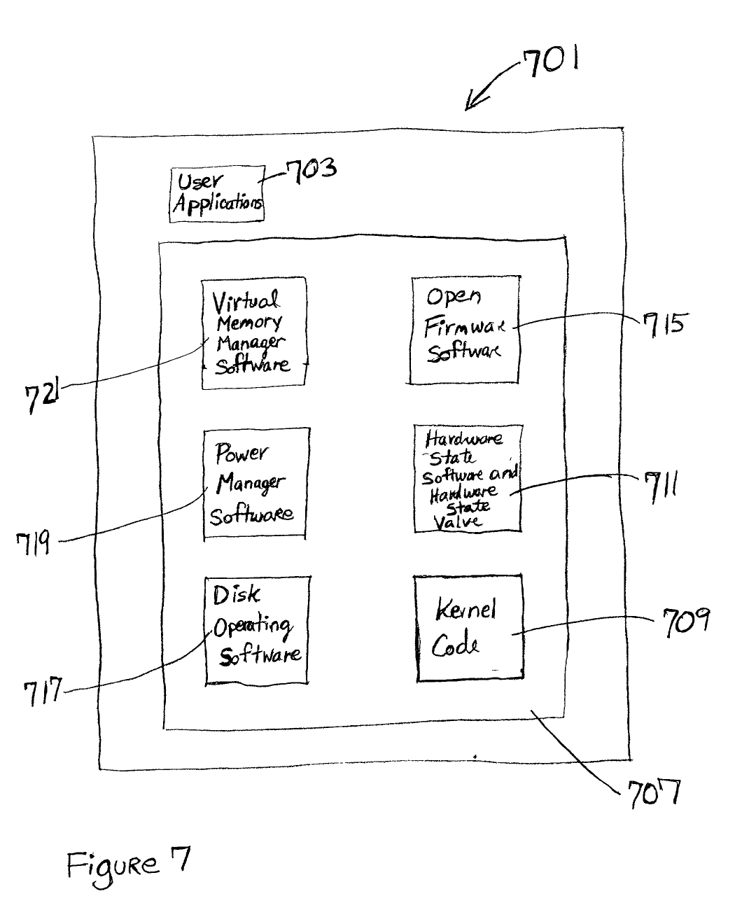

FIG. 7 shows an example of a computer readable media, which may be used with the data processing system according to one embodiment of the present invention. The computer readable media contains data and executable software which when executed in the data processing system such as a digital processing system cause the system to perform the various methods of the present invention. As noted above, this executable software and data may be stored in various places including for example the ROM 607, the volatile RAM 605, the non-volatile memory 606 and/or the cache 604. Portions of this software and/or data may be stored in any one of these storage devices. The media 701 for example may be primarily the volatile RAM 605 and the non-volatile memory 606 in one embodiment. The user applications 703 represent software applications, which are executing on the computer system, such as a word processing application or a spreadsheet application, an Internet web browser application, or an ambient-aware headset application, such as a headset interface application. The operating system 707 includes the Open Firmware software 715 which may be stored in the ROM 607 and loaded into RAM 605 at boot up. The hardware state software and hardware state value 711 is the software which generates the hardware state value. The kernel code 709 represents the kernel of the operating system and performs numerous tasks. The virtual memory manager software 721 controls the virtual memory process. This typically involves maintaining a map of page data which represents the state of data in all the virtual memory which includes the physical RAM such as volatile RAM 605 and a portion of the non-volatile memory 606 which has been designated as part of the virtual memory of the system. The virtual memory manager software will be performing conventional virtual memory processes as is known in the art. The power manager software 719 performs the various power managing operations such as notifying applications and the system and drivers of changes to the power consumption state of the system. The software may also monitor the state of a computer battery to determine whether sufficient power exists to continue to operate and displays alerts to the user indicating the status of the battery and the power status of the system. The disk operating system software 717 performs the conventional functions of a disk operating system. This typically includes controlling the operation of a hard disk drive which in many examples is the non-volatile memory 606 which serves as a virtual memory for the volatile RAM 605.

It will be further appreciated that the instructions represented by the blocks in FIGS. 3 and 4 are not required to be performed in the order illustrated, and that all the processing represented by the blocks may not be necessary to practice the invention.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

What is claimed is:

1. An apparatus comprising:
a mixer coupled to an external audio source to receive an external audio signal;
the mixer coupled to an ambient audio source to receive an ambient audio signal;
the mixer to mix the external audio signal and the ambient audio signal according to a specified relationship; and
a speaker coupled to the mixer to emit the external audio signal and the ambient audio signal into an ear canal of a user after the external audio signal and the ambient audio signal have been mixed by the mixer.

2. The apparatus of claim 1, wherein the ambient audio source, the mixer, and the speaker are an integrated assembly.

3. The apparatus of claim 1, further comprising a blocker coupled to the speaker to prevent entrance of the ambient audio signal that has not been mixed by the mixer from entering the ear canal of the user.

4. The apparatus of claim 1, further comprising a user audio preference interface (UAPI) coupled to the mixer to receive an audio preference from the user, the audio preference being used to determine the specified relationship.

5. The apparatus of claim 1, further comprising a noise filter coupled to the ambient audio source to filter noise from the ambient audio signal.

6. The apparatus of claim 1, further comprising a hearing compensator coupled to the mixer to compensate for a hearing defect of the user.

7. The apparatus of claim 1, the mixer further comprises a digital signal processor (DSP) to process the ambient audio signal and the external audio signal digitally.

8. The apparatus of claim 1, wherein the external audio source is selected from the group consisting of:
a telephone;
an audio playing device; and
a personal electronic device.

9. The apparatus of claim 1, wherein the ambient audio source is a microphone to capture ambient sound.

10. The apparatus of claim 1, further comprising a second microphone coupled to the external audio source for transmitting an outgoing audio signal from the user to the external audio source.

11. A method comprising:
receiving an external audio signal from an external audio source;
receiving an ambient audio signal;
mixing the external audio signal and the ambient audio signal according to a specified relationship; and
emitting the external audio signal and the ambient audio signal into an ear canal of a user after the external audio signal and the ambient audio signal have been mixed according to the specified relationship.

12. The method of claim 11, further comprising blocking entrance to the ear canal of the user by the ambient audio signal that has not been mixed according to the specified relationship.

13. The method of claim 11, wherein the specified relationship is predetermined by a preference input by the user.

14. The method of claim 11, further comprising filtering noise from the ambient audio signal.

15. The method of claim 14, wherein the filtering is performed according to a preference input by the user.

16. The method of claim 11, further comprising compensating for a hearing defect of the user.

17. The method of claim 16, wherein the compensating is performed according to a preference input by the user.

18. The method of claim 16, wherein the compensating is performed according to a preference input by a medical professional.

19. The method of claim 11, further comprising transmitting an outgoing audio signal from the user to the external audio source.

20. A system comprising:
an ambient-aware headset to receive an external audio signal from an external audio source, receive an ambient audio signal from an ambient audio source, mix the external audio signal and the ambient audio signal according to a specified relationship, and emit the external audio signal and the ambient audio signal into an ear canal of a user after the external audio signal and the ambient audio signal have been mixed according to the specified relationship; and
the external audio source communicatively coupled to the headset to receive the external audio signal from a communication apparatus and to provide the external audio signal to the headset.

21. The system of claim 20, further comprising a microphone communicatively coupled to the external audio source and the headset to transmit an outgoing audio signal from the user to the external audio source, the external audio source communicating the outgoing audio signal to the communication apparatus.

22. The system of claim 20, further comprising a user interface terminal communicatively coupled to the communication apparatus and the headset to store audio data and to relay the audio data between the communication apparatus and the headset.

23. A system comprising:
a processing unit:
a memory coupled to the processing unit through a bus; and
an ambient-aware headset interface process executed from the memory by the processing unit to receive an audio data signal from a communication apparatus and to provide the audio data signal to an ambient-aware headset; the headset receiving the audio data signal, receiving an ambient audio signal, mixing the audio data signal and the ambient audio signal according to a specified relationship, and emitting the audio data signal and the ambient audio signal into an ear canal of a user after the audio data signal and the ambient audio signal have been mixed according to the specified relationship

24. The system of claim 23, wherein the communication apparatus is selected from the group comprising:
a wireless telephone signal transmission tower; and
a wireless audio data signal transmission tower.

25. The system of claim 23, further comprising a microphone communicatively coupled to the headset to transmit an outgoing audio signal from the user to the communication device.

26. A machine-readable medium having stored thereon a plurality of instructions, which if executed by a machine, cause the machine to perform a method comprising:
receiving an external audio signal from an external audio source;
receiving an ambient audio signal;
mixing the external audio signal and the ambient audio signal according to a specified relationship; and
emitting the external audio signal and the ambient audio signal into an ear canal of a user after the external audio signal and the ambient audio signal have been mixed according to the specified relationship.

27. The machine-readable medium of claim 26, wherein the method further comprises blocking entrance to the ear canal of the user by the ambient audio signal that has not been mixed according to the specified relationship.

28. The machine-readable medium of claim 26, wherein the method further comprises at least one of the group consisting of:
filtering noise from the ambient audio signal; and
compensating for a hearing defect of the user.

29. The machine-readable medium of claim 26, wherein the specified relationship is predefined by the user.
